(19) United States
(12) Patent Application Publication
(10) Pub. No.: US 2006/0211911 A1
(43) Pub. Date: Sep. 21, 2006

(54) Vaginal insert

(76) Inventors: Shu-Wen Jao, Taipei (TW); Wei Lee, Taipei (TW)

Correspondence Address: TROXELL LAW OFFICE PLLC, SUITE 1404, 5205 LEESBURG PIKE, FALLS CHURCH, VA, 22041, US

(21) Appl. No.: 11/082,822

(22) Filed: Mar. 18, 2005

(51) Int. Cl. A61F 2/00

(52) U.S. Cl. 600/29

(57) ABSTRACT

Disclosed is a vaginal insert, which has a head for the holding of the user's hand, and a cylindrical front projection extending from the head for insertion into the vaginal of a rectocele patient to support the weakened lower vaginal wall in order to improve stool evacuation.

### BACKGROUND OF THE INVENTION

1. Field of the Invention

The present invention relates generally to a vaginal insert and more particularly, to such a vaginal insert that is insertable into the vaginal to support the weakened lower vaginal wall in order to improve the consitupation caused by rectocele.

2. Description of the Related Art

A rectocele is a bulge of the front wall of the rectum into the vagina. The rectal wall may be thinned and weak, and it may balloon out in the vagina when you push down to have a bowl movement. Most rectoceles occur in women where the front wall of the rectum is up against the back wall of the vagina. This area is called the rectovaginal septum and may be a weak area in the female anatomy. Other structures may also push into the vagina. The bladder bulging into the vagina is called a cystocele. The rectum bulging into the vagina is termed a rectocele. A history of constipation and straining with bowel movements, or hysterectomy may contribute to the development of a rectocele. Commonly, these problems develop with age but they may occasionally occur in younger women or in those that have not delivered children. Rectocele may cause constipation due to incomplete evacuation stool. Many women need digitation through vagina to help defecation. But digitation is not convenient and also may cause infection. So, we design the vaginal insert, which can be repeat clean and used in each defecation.

### SUMMARY OF THE INVENTION

The present invention has been accomplished under the circumstances in view. It is the main object of the present invention to provide a vaginal insert for a rectocele patient to support the weakened lower wall of the vagina.

According to one aspect of the present invention, the vaginal insert comprises a head for the holding of the user's hand, and a cylindrical front projection extending from said head for insertion into the vaginal of a rectocele patient to support the weakened lower vaginal wall. The cylindrical front projection has a smooth outer surface and a free end terminating at an outwardly curved arched portion. According to another aspect of the present invention, the head comprises an expanded holder portion formed integral with one end of the cylindrical front projection, and a cap member fixedly fastened to the expanded rear holder portion by an ultrasonic sealing apparatus.

### BRIEF DESCRIPTION OF THE DRAWINGS

FIG. 1 is an elevational view of a vaginal insert according to the present invention.

FIG. 2 is an exploded view of the vaginal insert according to the present invention.

FIG. 3 is a sectional view of the vaginal insert according to the present invention.

FIG. 4 is an elevational view of an alternate form of the vaginal insert according to the present invention.

FIG. 5 is a schematic drawing showing the status before use of the present invention.

FIG. 6 is a schematic drawing showing the status in use of the present invention.

### DETAILED DESCRIPTION OF THE INVENTION

A vaginal insert is a hollow device having a cylindrical front projection 11 and a head 20 at the rear end of the cylindrical front projection 11 for the holding of the hand. The cylindrical front projection 11 has a smooth outer surface and is terminated at an outwardly curved arched portion 13.

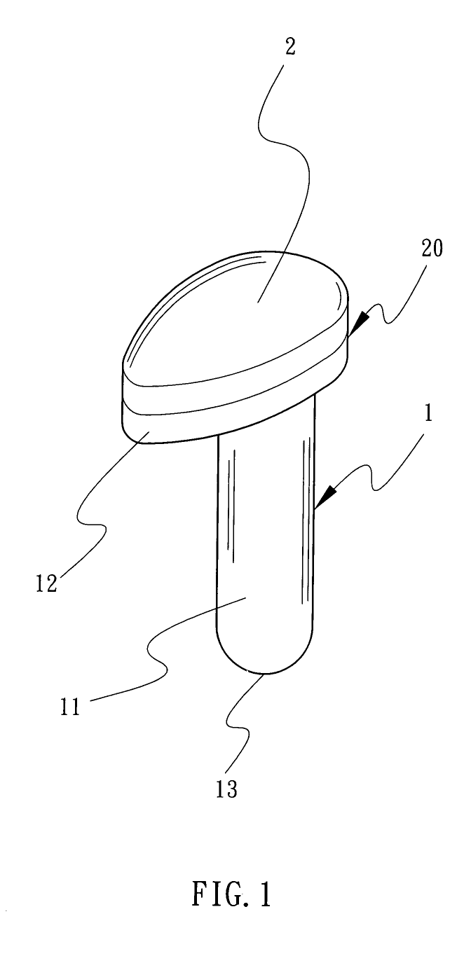

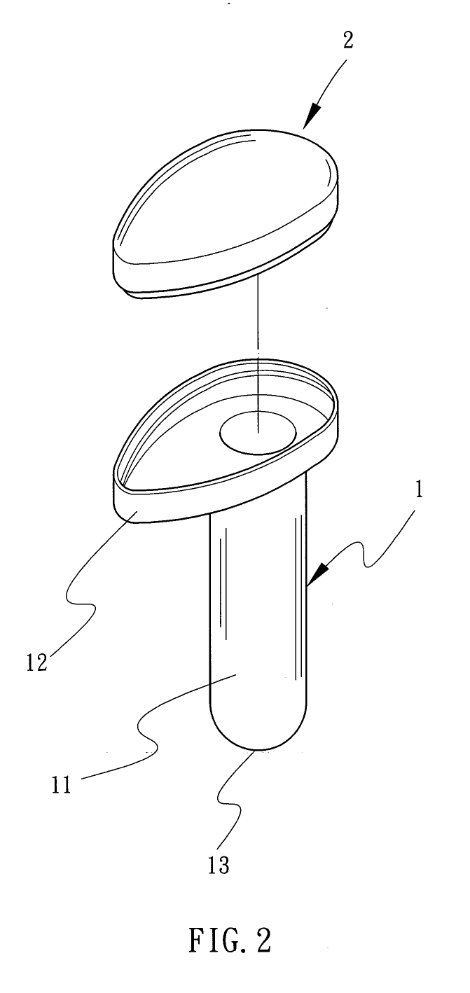

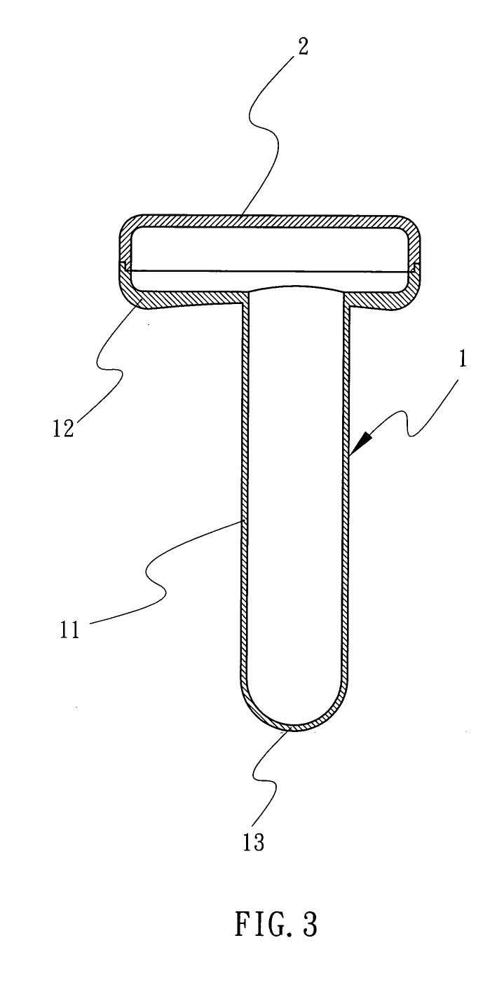

Referring to FIGS. 1˜3, the vaginal insert is shown formed of two separate members, i.e. the hollow cylindrical member 1 and a cap member 2.

The hollow cylindrical member 1 has a cylindrical front projection 11 and an expanded rear holder portion 12. The cap member 2 is fixedly fastened to the expanded rear holder portion 12 by an ultrasonic sealing apparatus (or by means of the application of an adhesive or screw means), thereby forming with the expanded rear holder portion 12 the aforesaid head 20.

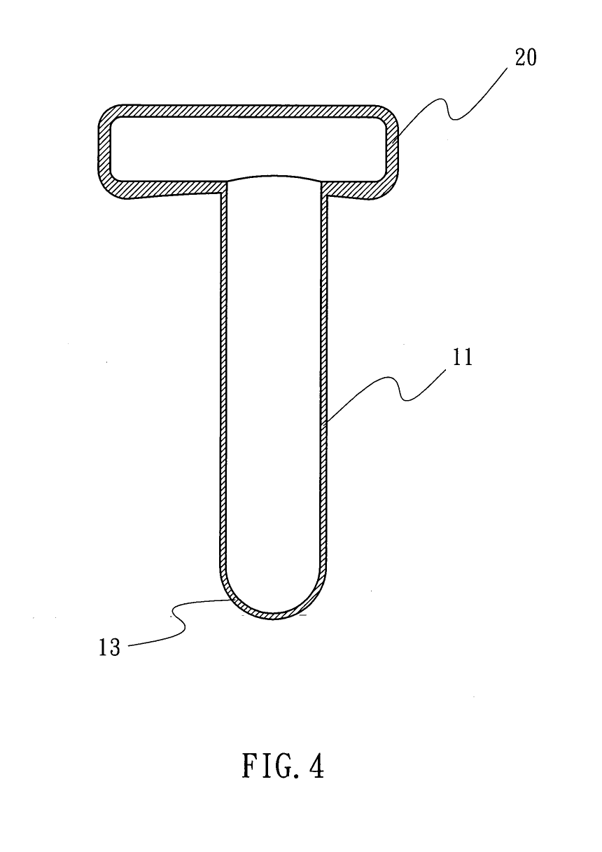

According to the embodiment shown in FIGS. 1˜3, the vaginal insert is formed of two separate members that are fastened together by an ultrasonic sealing apparatus (or by means of the application of an adhesive or screw means). FIG. 4 shows an alternate form of the present invention. According to this embodiment, the vaginal insert is directly injection-molded from plastics, having a cylindrical front projection 11 and a head 20 formed integral with the rear end of the cylindrical front projection 11 for the holding of the hand. The cylindrical front projection 11 has a smooth outer surface and is terminated at an outwardly curved arched portion 13.

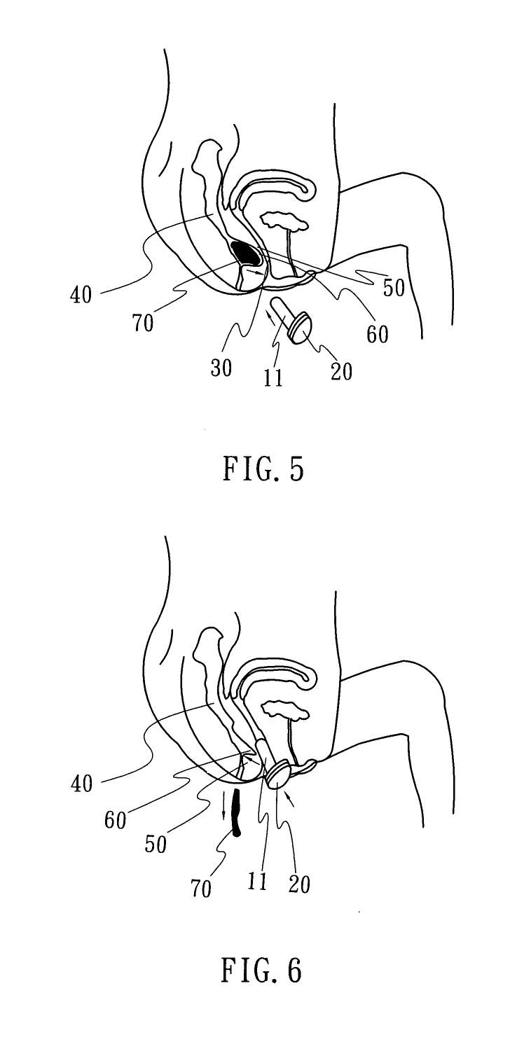

Referring to FIG. 5, a rectocele patient has the rectovaginal septum 50 between the vagina 30 and the rectum 40 in a loose status, and the weakening of the lower vaginal wall causes the rectum 40 to bulge into the vagina 30, thereby forming a recesses area 60 where excrement 70 is accumulated.

Referring to FIG. 6, the user holds the head 20 and then inserts the cylindrical front projection 11 into the vagina 30 to push the rectovaginal septum 50 outward against the rectum 40, thereby guiding the accumulated excrement 70 back to the rectum 40.

As indicated above, the invention provides a vaginal insert that helps rectocele patients easy to evacuation stool. The vaginal insert is re-washable for repeated use.

A prototype of vaginal insert has been constructed with the features of FIGS. 1˜6. The vaginal insert functions smoothly to provide all of the features discussed earlier.

Although particular embodiments of the invention have been described in detail for purposes of illustration, various modifications and enhancements may be made without departing from the spirit and scope of the invention. Accordingly, the invention is not to be limited except as by the appended claims.

What is claimed is:

1. A vaginal insert comprising a head for the holding of the user's hand, and a cylindrical front projection extending from said head for insertion into the vaginal of a female, said cylindrical front projection having a smooth outer surface and a free end terminating at an outwardly curved arched portion.

2. The vaginal insert as claimed in claim 1, which is a hollow member made in integrity.

3. The vaginal insert as claimed in claim 1, which is formed of two separate members that are fastened together by means of one of a group of methods including ultrasonic sealing, the application of an adhesive, screw means.

4. The vaginal insert as claimed in claim 1, wherein said head comprises an expanded holder portion formed integral with one end of said cylindrical front projection, and a cap member fixedly fastened to said expanded rear holder portion by a ultrasonic sealing apparatus.
